(19) United States
(12) Patent Application Publication
(10) Pub. No.: US 2005/0209703 A1
(43) Pub. Date: Sep. 22, 2005

(54) Surgically implantable prosthetic system

(76) Inventor: Barry M. Fell, Hummelstown, PA (US)

Correspondence Address: BROOKS KUSHMAN P.C., 1000 TOWN CENTER, TWENTY-SECOND FLOOR, SOUTHFIELD, MI, 48075, US

(21) Appl. No.: 11/117,838

(22) Filed: Apr. 29, 2005

Related U.S. Application Data
(63) Continuation-in-part of application No. 10/232,608, filed on Aug. 30, 2002.
Application No. 10/232,608 is a continuation of application No. 09/934,364, filed on Aug. 21, 2001, now abandoned.
Application No. 09/934,364 is a continuation-in-part of application No. 09/664,939, filed on Sep. 19, 2000, now Pat. No. 6,558,421.
Application No. 09/664,939 is a continuation of application No. 09/297,943, filed on May 10, 1999, now Pat. No. 6,206,927.

(51) Int. Cl. A61F 2/38

(52) U.S. Cl. 623/20.33; 623/14.12

(57) ABSTRACT

A prosthetic system is provided for implantation into a body joint, such as a knee joint compartment between a femoral condyle and its corresponding tibial plateau. The prosthetic system includes a tibial component arranged to be fixed to the tibial plateau and having a top face including a generally flat surface and an opposed bottom face. The prosthetic system further includes a mensical component having a generally elliptical shape in plan, the mensical component having a top face and an opposed bottom face including a generally flat surface. The mensical component bottom face is arranged to engages the tibial component top face to form the assembled prosthetic system, where the mensical component is movable with respect to the tibial component.

### CROSS-REFERENCE TO RELATED APPLICATIONS

This application is a continuation-in-part of U.S. application Ser. No. 10/232,608, filed Aug. 30, 2002, which is a continuation of U.S. application Ser. No. 09/934,364, filed Aug. 21, 2001, now abandoned, which is a continuation-in-part of U.S. application Ser. No. 09/664,939, filed Sep. 19, 2000, now U.S. Pat. No. 6,558,421, which is a continuation of U.S. application Ser. No. 09/297,943, filed May 10, 1999, now U.S. Pat. No. 6,206,927, which is the National Stage of International Application No. PCT/US99/07309, filed Apr. 2, 1999, wherein all of the above-identified applications are incorporated by reference herein.

### BACKGROUND OF THE INVENTION

1. Field of the Invention

The present invention relates to a prosthetic system which may be surgically implanted within a body joint, such as within the knee joint compartment, and a method for implantation thereof.

2. Background Art

Articular cartilage and meniscal cartilage provide the mobile weight bearing surfaces of the knee joint. Damage to these surfaces is generally due to genetic predisposition, trauma, and/or aging. The result is usually the development of chondromalacia, thinning and softening of the articular cartilage, and degenerative tearing of the meniscal cartilage. Various methods of treatment are available to treat these disease processes. Each option usually has specific indications and is accompanied by a list of benefits and deficiencies that may be compared to other options.

The healthy knee joint has a balanced amount of joint cartilage across the four surfaces of this bicompartmental joint (medial femoral condyle, medial tibial plateau, lateral femoral condyle, and lateral tibial plateau). In patients with osteoarthritis, the degenerative process typically leads to an asymmetric wear pattern that leaves one compartment with significantly less articular cartilage covering the weight bearing areas of the tibia and femur than the other compartment. Most commonly, the medial compartment of the knee joint is affected more than the lateral compartment.

As the disease progresses, large amounts of articular cartilage are worn away. Due to the asymmetric nature of the erosion, the alignment of the mechanical axis of rotation of the femur relative to the tibia becomes tilted down towards the compartment which is suffering the majority of the erosion. The result is a varus (bow-legged) deformity in the case of a medial compartment disease predominance, or a valgus (knock-kneed) deformity in the case of lateral compartment disease predominance. Factors such as excessive body weight, previous traumatic injury, knee instability, the absence of the meniscus, and genetic predisposition all affect the rate of the disease.

Osteoarthritis is usually defined in stages of Grade I through V, with Grade III revealing significant articular cartilage loss, Grade IV revealing some eburnation of the subchondral bone, and Grade V detailing both significant articular loss and bone loss. The disease manifests itself as periodic to continuous pain that can be quite uncomfortable for the patient. The cause of this pain is subject to many opinions but it is apparent that, as the joint compartment collapses, the collateral ligament on the side of the predominant disease becomes increasingly slack and the tibial and femoral axes move, for example, from a varus to a valgus condition. This increases the stress on the opposing collateral ligament as well as the cruciate ligaments, and shifts the load bearing function of this bicompartmental joint increasingly towards the diseased side. This increasing joint laxity is suspected of causing some of the pain one feels. In addition, as the bearing loads are shifted, the body responds to the increased loading on the diseased compartment with an increased production of bony surface area (osteophytes) in an attempt to reduce the area unit loading. All of this shifting of the knee component geometry causes a misalignment of the mechanical axis of the joint. This misalignment causes an increase in the rate of degenerative change to the diseased joint surfaces, causing an ever-increasing amount of cartilage debris to build up in the joint, and further causing joint inflammation and subsequent pain.

Currently, there is a void in options used to treat the relatively young patient with moderate to severe chondromalacia involving mainly one compartment of the knee. Current treatments include NSAIDS, cortisone injections, hyaluronic acid (HA) injections, and arthroscopic debridement. Some patients cannot tolerate or do not want the risk of potential side effects of NSAIDS. Repeated cortisone injections actually weaken articular cartilage after a long period of time. HA has shown promising results, but is only a short term solution for pain. Arthroscopic debridement alone frequently does not provide long lasting relief of symptoms.

Unfortunately, the lack of long term success of these treatments leads to more invasive treatment methods. Osteochondral allografts and microfracture techniques are indicated for small cartilage defects that are typically the result of trauma. These procedures are not suitable for addressing large areas of degeneration. In addition, osteochondral allografts can only be used to address defects on the femoral condyle, as tibial degeneration cannot be addressed with this technique. High tibial osteotomy (HTO) corrects the varus malalignment between the tibia and the femur but, because it is performed below the joint line, it does not fill the cartilage void or re-tension the medial collateral ligament (MCL). Removing bone and changing the joint line does not complicate the conversion to total knee arthroscopy (TKA). However, an HTO does leave a hard sclerotic region of bone which is difficult to penetrate, making conversion to a total knee replacement technically challenging.

Currently, patients with large tibial defects require replacement of the existing surfaces with materials other than articular cartilage, namely with uni-condylar (UKR) or total (TKR) knee replacements. Unfortunately, these procedures resect significant amounts of bone (typically 7-9 mm), and primary (first arthroplasty performed on the joint) procedures have a functional life span of only 5-10 years, such that younger patients will likely require revision surgery as they age. Furthermore, the amount of bone loss that is inherent in a UKR or TKR makes a revision (secondary) procedure much more difficult in the future as even more bone must be removed. Revision knee replacement surgery is usually extensive and results in predictably diminished mechanical life expectancy. Therefore, it is best to delay these types of bone resecting surgeries as long as possible.

The only true solution is to rebuild the defective joint by “filling” the joint space with more articular bearing material through a complete resurfacing of the existing femoral condyle and tibial plateau. By replacing the original cartilage to its pre-diseased depth, the joint mechanical axis alignment is restored to its original condition. Unfortunately, these natural articular materials and surgical technology required to accomplish this replacement task do not yet exist. The alternative method is to fill the joint space with a spacer that replaces the missing articular materials.

### BRIEF DESCRIPTION OF THE DRAWINGS

FIG. 1 is a top perspective view of a tibial component according to the present invention;

FIG. 2 is a top perspective view of a tibial component according to another aspect of the present invention;

FIG. 3 is a top perspective view of a meniscal component according to the present invention;

FIG. 4 is a bottom perspective view of the meniscal component of FIG. 3;

FIG. 5A is a cross-sectional view of the mensical component of FIG. 3 taken along line A-A;

FIG. 5B is a cross-sectional view of the mensical component of FIG. 3 taken along line B-B;

FIG. 6 is a top perspective view of a mensical component according to another aspect of the present invention;

FIG. 7 is a bottom perspective view of the meniscal component of FIG. 6;

FIG. 8 is a cross-sectional view of the meniscal component of FIG. 6 taken along line C-C;

FIG. 9 is a perspective view of a prosthetic system according to the present invention comprising the tibial component of FIG. 1 and the mensical component of FIG. 3;

FIG. 10 is an end elevational view of the prosthetic system of FIG. 9;

FIG. 11 is a top perspective view of a prosthetic system according to the present invention comprising the tibial component of FIG. 2 and the mensical component of FIG. 3;

FIG. 12 is an end elevational view of the prosthetic system of FIG. 11;

FIG. 13 is an end elevational view of a prosthetic system according to the present invention comprising the tibial component of FIG. 1 and the mensical component of FIG. 6;

FIG. 14 is an end elevational view of a prosthetic system according to the present invention comprising the tibial component of FIG. 2 and the mensical component of FIG. 6;

FIG. 15 is a top plan view of the mensical component of FIG. 3 with reference to a coordinate system; and

FIG. 16 illustrates an exemplary placement of a prosthetic system according to the present invention in a knee joint.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale, and some features may be exaggerated or minimized to show details of particular components. Therefore, specific structural and functional details disclosed herein are not to be interpreted as limiting, but merely as a representative basis for teaching one skilled in the art to variously employ the present invention.

The prosthetic system according to the present invention is a unicompartmental system suitable for minimally invasive, surgical implantation into a knee compartment. The knee joint compartment is defined by the space between a femoral condyle and the respective tibial plateau, in which a portion of the natural meniscus is ordinarily located. By effectively replacing worn articular material, the prosthetic system of the present invention may restore the normal joint alignment and may provide a smooth bearing surface against which the femoral condyle can articulate. Further, it can minimize articulation of the femoral condyle against the tibial plateau, thereby preventing further degradation of the tibial surface. By occupying the joint space and retensioning the collateral ligaments, the prosthetic system according to the present invention can improve joint stability and may restore the limb to a more normal mechanical alignment.

By the term “unicompartmental” it is meant that the prosthetic system of the present invention is suitable for implantation into but one medial or lateral compartment defined by the space between a femoral condyle and its associated tibial plateau. In other words, the present system is not a bicompartmental system which, in one rigid unit, could be inserted into both of the two femoral condyle/tibial plateau compartments. In many, if not most, cases a prosthetic system will be inserted into one compartment only, either the medial or lateral compartment. Most often, it will be the medial compartment as the meniscus and associated articular surfaces in the medial compartment are most subject to wear and damage. However, it is possible to insert two separate prosthetic systems according to the present invention into the medial and lateral compartments of the same knee, or to use two such prosthetic systems that are mechanically, but non-rigidly, linked.

While the prosthetic system according to the present invention is shown and described herein as being implanted in a knee joint, it is understood that the prosthetic system could be utilized in body joints other than the knee such as, but not limited to, the hip, shoulder, wrist, ankle, or elbow.

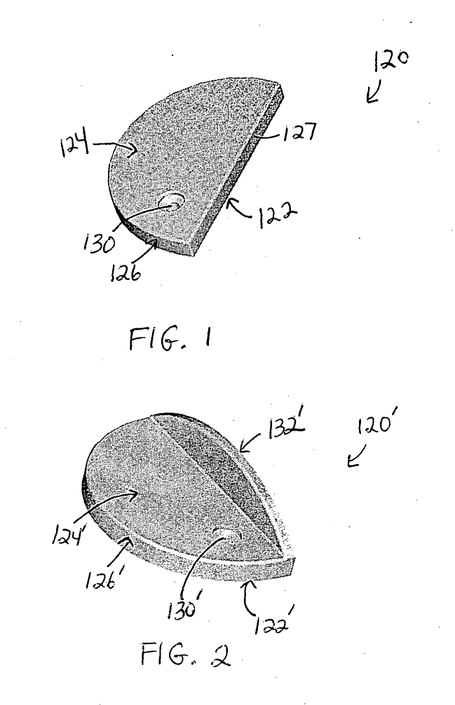

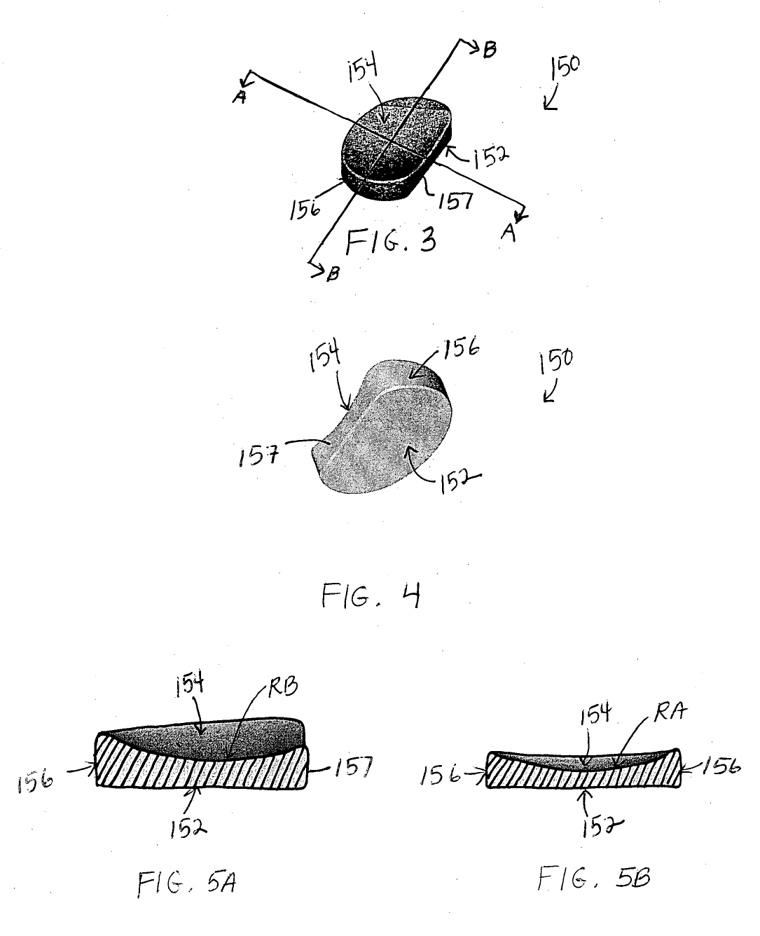

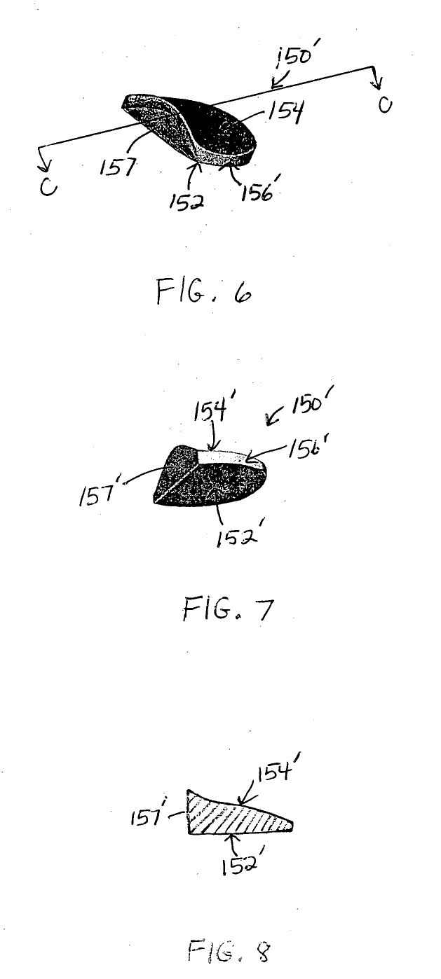

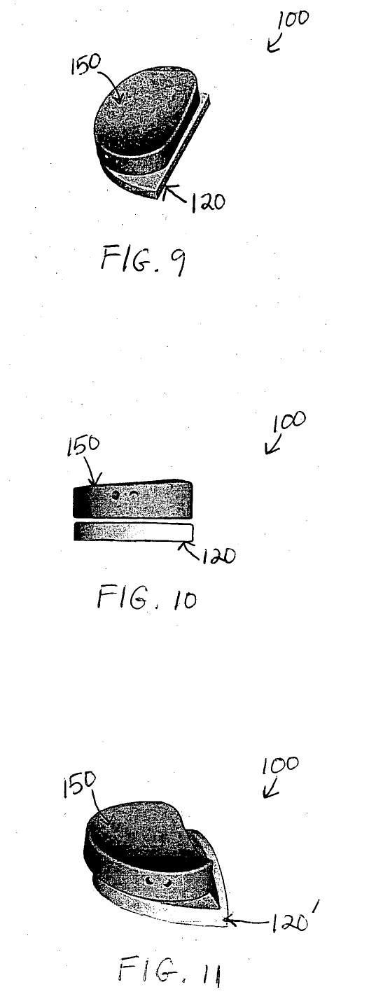

Turning first to FIGS. 1-8, components of the prosthetic system according to the present invention are illustrated. The prosthetic system includes a bottom, tibial component 120, 120′ (FIGS. 1 and 2) and a top, mensical component 150, 150′ (FIGS. 3-8). With reference to FIG. 1, tibial component 120 can comprise a generally semicircular shape in plan and may be arranged to be fixed to the tibial plateau. Of course, it is understood that other shapes of tibial component 120 compatible with implantation in a specified body joint compartment are fully contemplated in accordance with the present invention. Tibial component 120 includes a bottom face 122 and a top face 124, wherein top face 124 may include a generally flat surface, such as to facilitate movement of a mensical component with respect to tibial component 120 as described below. Bottom face 122 may be designed to generally match the contour of the tibial plateau following any necessary bone resection or resurfacing. A peripheral edge 126 extends between bottom face 122 and top face 124, wherein peripheral edge 126 may be generally rounded as shown and may include a generally straight first side 127. Tibial component 120 can include an aperture 130 through which a screw or other fastener can be introduced for securing tibial component 120 to the tibial plateau. Of course, it is understood that other means of fixation, such as mating ridges/depressions, porous areas to accommodate tissue regrowth, or cement fixation, are also fully contemplated in accordance with the present invention.

According to another aspect of the present invention, the tibial component 120′ depicted in FIG. 2 includes a guide 132′ extending upwardly from top face 124′, such as at the approximately 90° angle shown herein, which can serve to constrain movement of the mensical component in the medial-lateral direction. Reference numerals for tibial component 120′ are the same as those for tibial component 120 except for the addition of a prime (′) designation. When implanted in a patient's knee compartment, side 127 of tibial component 120 or guide 132′ of tibial component 120′ is typically proximate to the tibial spine. While guide 132′ is not limited to any particular height or configuration, guide 132′ may be provided at a height sufficient to allow support of the mensical component without being significantly greater in height than the mensical component when the tibial and mensical components are assembled to form the prosthetic system 100.

While not shown herein, it is understood that the prosthetic system according to the present invention can be used in conjunction with a femoral component of any configuration, wherein the femoral component is affixed to the femur as is known in the art.

Turning next to FIGS. 3-5, a mensical component 150 according to the present invention is illustrated. According to one aspect of the present invention, mensical component 150 can include a generally elliptical shape in plan and includes a bottom face 152 and a top face 154 with a peripheral edge 156 extending therebetween, wherein peripheral edge 156 may be rounded. According to one aspect of the present invention, peripheral edge 156 includes at least one generally straight side 157, such as when used with tibial component 120′ in order to facilitate movement of mensical component 150 along guide 132′ in the anterior-posterior direction. Top face 154 can act as the bearing surface for the femoral condyle or a femoral component and bottom face 152 may be arranged to engage top face 124 of tibial component 120, 120′. Bottom face 152 may include a generally flat surface, such as to facilitate sliding easily along tibial component 120 and creating maximum surface area contact therewith. It is understood that mensical component bottom face 152 need not be totally supported by tibial component top face 124, 124′ as long as mensical component 150 is sufficiently supported to allow movement of bottom face 152 along top face 124, 124′.

Meniscal component top face 154 can have a contour which is generally non-conformal to a surface of the femoral condyle or of a femoral component. Alternatively, top face 154 of mensical component 150 can include a contour which is generally conformal to the femoral surface (either femoral condyle or femoral component). More specifically, top face 154 can include a generally concave surface as illustrated in FIGS. 3-5 (typically for a medial compartment implantation). However, it is understood that other contours of top face 154 are fully contemplated in accordance with the present invention.

If a prosthetic system is desired for the lateral compartment of the knee, mobile mensical component 150′ shown in FIGS. 6-8 can be selected. Top face 154′ can include a generally convex surface for proper ligament tensioning, and can have a contour which is generally conformal to the surface of the femoral condyle or a femoral component. With this exception, it is understood that the foregoing description of mensical component 150 applies equally well to mensical component 150′, wherein like features are represented by like reference numerals except for the addition of a prime (′) designation.

It is understood that the term “generally elliptical” as used herein is intended to include the shape of tibial components 120, 120′ and mensical components 150, 150′ described herein as well as all construction methods which yield a generally planar shape which is longer in one direction than in the transverse direction and has rounded corners, and that the tibial and mensical components of the present invention are not otherwise limited to any particular shape. Furthermore, it is understood that the terms “concave” and “convex” as used herein are not restricted to describing surfaces with a constant radius of curvature, but rather are used to denote the general appearance of the surfaces.

Meniscal components 150, 150′ according to the present invention are preferably translatable but self-centering. By “translatable” it is meant that during natural articulation of the knee joint the mensical component is allowed to move or change its position, such that articulation of the knee results in a modest amount of lateral/medial and anterior/posterior translation of the mensical component 150, 150′ relative to the tibial component 120, 120′. The term “self-centering” means that upon translation from a first position to a second position during knee articulation, mensical component 150, 150′ will return to substantially its original position as the articulation of the knee joint is reversed and the original knee position is reached. Thus, mensical component 150, 150′ will not progressively “creep” towards one side of the compartment in which it is located, but rather the mensical component 150, 150′ is shaped such that its contour and the natural articulation of the knee exerts a restoring force on the mensical component 150, 150′. The angle of attack of the femoral condyle against the mensical component 150, 150′ will ensure that the mensical component reversibly translates during articulation, maintaining the mensical component, on average, in the same location for any given degree of knee articulation. In order to ensure the ability of the mensical component to “self-center,” adequate tension of the cruciate ligaments should be maintained.

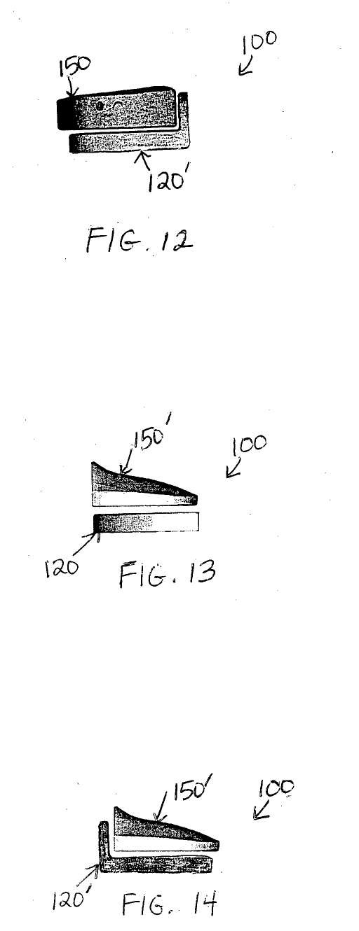

Turning now to FIGS., assembled prosthetic systems 100 according to the present invention are illustrated. Specifically, FIGS. 9-10 illustrate a prosthetic system 100 comprising tibial component 120 and mensical component 150, FIGS. 11-12 illustrate a prosthetic system 100 comprising tibial component 120 and mensical component 150, FIG. 13 illustrates a prosthetic system comprising tibial component 120 and meniscal component 150′, and FIG. 14 illustrates a prosthetic system comprising tibial component 120′ and 150′. In practice, tibial component 120, 120′ can be fixed to the tibial plateau with first side 127, 127′ proximate to the tibial spine, and subsequently mensical component 150, 150′ can be inserted into the knee joint compartment and seated on tibial component 120, 120′.

As shown, tibial components 120, 120′ can be used equally as easily with either mensical component 150 or 150′, as well as with any other mensical component having the appropriate sizing and construction for mating with tibial components 120, 120′. This modularity of prosthetic system 100 of the present invention may allow the physician to implant a standard tibial component while providing flexibility in the selection of a mensical component that is best suited for each individual patient. Furthermore, should it be necessary to change the mensical component selection following implantation, the entire prosthetic system 100 need not be removed, but instead only the mensical component, as the tibial component can remain fixed to the tibial plateau. This capability of prosthetic system 100 of the present invention can reduce the recovery time and trauma associated with such a procedure.

Contrary to most devices which are composed of soft, compliant material designed to assume the function of the natural meniscus which they replace, the tibial and mensical components of prosthetic system 100 of the present invention may comprise a relatively hard, relatively high modulus, preferably low friction material. Suitable materials include, for example, metals such as steel or titanium, metal alloys such as those described in U.S. Pat. Nos. 3,989,517; 5,368,659; and 5,618,359 (LiquidMetal, Inc.), ceramics, and reinforced and non-reinforced thermoset or thermoplastic polymers. Of course, the tibial and mensical components of prosthetic system 100 need not be formed of the same material. For example, tibial component 120, 120′ could be hard, whereas a conformal mensical component 150, 150′ may be constructed from a relatively lower modulus material to allow for some deformation. Furthermore, the tibial and mensical components of prosthetic system 100 need not be made only of a single material. Rather, the tibial and mensical components can each have areas of lower modulus material, and composite structures of steel/thermoplastic, steel/ceramic, ceramic/polymer, or the like may be used. It is understood that the term “hard” as used herein is used to describe a material that is sufficient to span defects in the tibia or femur without substantially deforming into the defects, allowing for the provision of recessed or non-contacting areas of the prosthetic system to encourage articular regeneration.

In greater detail, materials could include elastomeric polymers such as nylon, silicone, polyurethane, polypropylene, polyester, or the like, optionally fiber-reinforced, or viscous-elastic materials such as hydrogels, as well as other hydrophilic materials or hydrophobic materials. Polymers capable of containing living cells could also be utilized. Still other possible materials are those which can replicate the function of naturally occurring cartilage or meniscus such as the CSTI meniscal repair material that is the subject of numerous U.S. patents to Stone, for example, U.S. Pat. No. 5,158,574. A surface coating can be employed, such as for the reduction of friction between tibial component and mensical component, as well as for the reduction of friction between the mensical component and the femoral condyle. Generally, the areas of prosthetic system 100 expected to have the most wear due to either high stress or greater movement relative to the other component or the femoral condyle may be made of stronger, more abrasion resistant material than the remainder of prosthetic system 100 when composite structures are used. As such, it is understood that particular areas may be softer than the material used for constructing the majority of the tibial 120, 120′ and mensical components 150, 150′.

In accordance with the present invention, the tibial and mensical components of prosthetic system 100 may be manufactured so as to substantially contain, or have deposited thereon, a biologically or pharmaceutically active material such as, for example, one that promotes tissue regrowth, retards tissue degeneration, or decreases inflammation. This is particularly suitable when the tibial or mensical component functions to bridge a defective area of bone or articular cartilage. The active material can be provided in the form of a coating on tibial or mensical component, or can be contained within tibial or mensical component in the form of a solid, liquid, gel, paste, or soft polymer material. Such active materials may be designed to be delivered at once or in a timed-release manner. According to one aspect of the present invention, the area of the tibial or mensical component containing the active material may be configured to not actually contact, or minimally contacts, the damaged tissue.

As stated above, one purpose of the prosthetic system 100 of the present invention is to achieve a span-like effect to bridge areas of the femoral condyle and/or tibial plateau which have been damaged or have experienced tissue degeneration. If a soft and/or low modulus elastomer or thermoplastic were to be used for the entire prosthetic system as in prior art devices, not only would the load not be concentrated on healthy tissue, but damaged areas would also be subjected to static and dynamic loading and wear, thereby decreasing the opportunity for the body's natural regenerative capability to function. Under such circumstances, active materials will be rapidly dissipated and newly regenerated articular cartilage not having the necessary density or cohesiveness to withstand wear will quickly be eroded away. Rather than deforming as in prior art devices to distribute a load relatively equally on the mating femoral and tibial surfaces, prosthetic system 100 according to the present invention does not necessarily spread the load uniformly, but rather may redistribute the load to healthy tissue, spanning areas of imperfection and allowing inflamed, diseased, or other damaged areas to regenerate. Moreover, as regeneration proceeds, the regenerating tissue may assume a shape dictated by the shape of tibial component bottom face 122, 122′ and mensical component top face 154, 154′. Growth under these circumstances has the greatest potential for dense, ordered cartilage most closely replicating the original surface.

Much study has been dedicated to determine if any relationship exists in the normal human anatomy that would allow one to define the required dimensions of the prosthetic system for proper fit and function based on a single, easy to establish, measurable anatomic landmark. Based on a study of over 100 MRI's and 75×-rays of human subjects ranging from 15 to 87 years of age, a relationship was established between the anteroposterior radius of the most distal portion of the femoral condyle and the dimensions which control the geometric form of the prosthesis. The database revealed a range of femoral anteroposterior radii from 32 mm to 48 mm. However, it is known that the worldwide range is much larger because of genetic differences in the human anatomy.

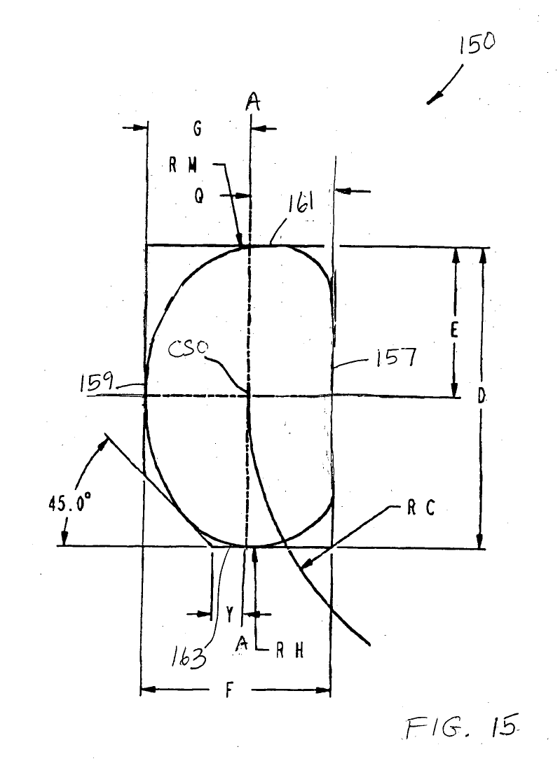

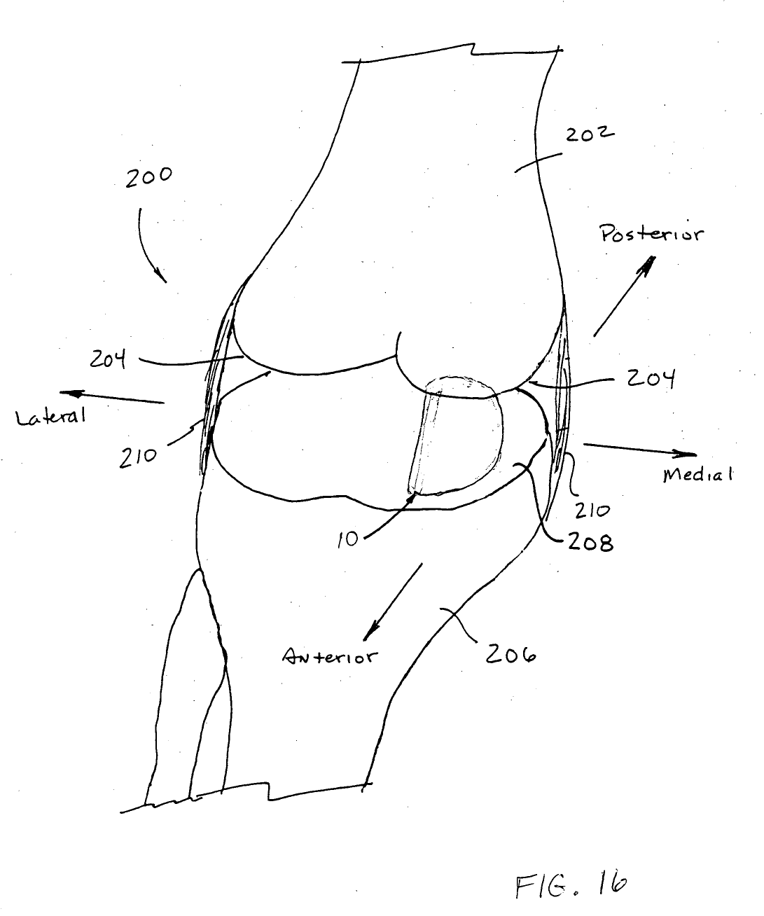

With reference now to FIG. 15, a coordinate system is superimposed, for example, on mensical component 150 according to the present invention. It has been found that a suitable size for the tibial and mensical components is defined by a minor axis F (distance from first side 157 to second side 159) and a major axis D (distance from first end 163 to second end 165) which are related by a ratio ranging from F=0.25D to 1.5D. The appropriate thickness of prosthetic system 100 can be determined by measuring the amount of joint space between the femoral and tibial joint surfaces when a minor amount of valgus (heels out, knees in) is applied to the knee. While the top and bottom faces of tibial components 120, 120′ may be generally lacking curvature, the top faces of mensical components 150 and 150′ each may define a femoral radius RA (see, for example, FIG. 5B). The relationship between femoral radius RA to other joint dimensions (femoral radius is the driving relationship to all other dimensions) according to one aspect of the present invention is as follows:
Medial-lateral radius RB=0.25RA to 1.0RA (see, for example, FIG. 5A)
Curve of anterior half of femoral radius RC=0.5RA to 2.0RA, posterior half is straight
Length D=0.6RA to 1.4RA
Posterior half E=0. IRA to 0.75RA
Width F=0.25RA to 1.5RA
Width from part center to medial edge G=0.096RA to 0.48RA
Anterior plan radius RH=0.16RA to 0.64RA
Posterior plan radius RM=0.16RA to 0.64RA
Width from part center to lateral edge Q=−0.32RA to 0.32RA
Location of transition from anterior radius to medial radius Y=−0.32RA to 0.32RA

Above, a negative value means that a dimension may extend to an opposite side of line A-A. Although mensical component 150 is depicted herein relative to the coordinate system, it is understood that the other mensical component 150′ and the tibial components 120, 120′ also include a second side opposite their first side defining the dimension F for each component and opposed first and second ends defining the dimension D for each component, and that the ranges provided above may apply equally well to these other meniscal and tibial components.

The actual shape of the components of prosthetic system 100 may be tailored to the individual. Individuals with high varus or valgus deformation due to wear, degeneration, or disease may require a prosthetic system (in particular, a mensical component) which is of considerably greater thickness over the portions where wear is most advanced. In youthful patients, where trauma-induced damage rather than severe wear or degeneration has occurred, differences in mensical component thickness may be more moderate. By the very nature of the ability to adjust for the lost articular material through the thickness of the prosthetic system of the present invention, the thickness adjustment may substantially eliminate the need for a functional meniscus as a bearing surface in a severely (Grade III or IV) degenerated knee. In these instances, the top face of the mensical component may reside significantly above the meniscal edge, and the meniscus may be completely unloaded. As the highest compressive loads in the knee occur with the knee substantially extended, the outer contours of the tibial and mensical components may be designed to substantially mate with the corresponding tibial and femoral surfaces when the knee is in full extension so that the high compressive loads can be distributed over large surface areas.

Generally speaking, each knee presents a different geometry of the respective femoral condyles and tibial plateaus. Even with respect to the right and left knees of a single individual, although bilateral symmetry dictates that the left and right knee components should be mirror images, this is often only an approximation. Thus, the shape of the affected femoral condyle and tibial plateau (while discussed herein in the singular, more than one pair of condyle(s)/plateau(s) may be involved), may be ascertained to determine the correct geometry of the prosthetic system 100 for a given patient. The tibial and mensical components according to the present invention provide a prosthetic system 100 which is modular, such that a specific mensical component can be selected for use with a more standard tibial component.

To implant a prosthetic system 100 that possesses the characteristics of the subject invention, the patient's knee joint may be examined by a non-invasive imaging procedure capable of generating sufficient information such that appropriately sized and shaped components may be selected. A variety of non-invasive imaging devices may be suitable, for example magnetic resonance imaging (MRI), X-ray devices and the like.

In one method, MRI or other non-invasive imaging scans, optionally coupled with exterior measurements of the dimensions of the relevant tibial and femoral portions including the surface of the articular cartilage of the tibia and femur, may be used to establish a library of tibial and mensical components whose size and geometry differ according to the age and size of the patient, the patient's genetic make-up, and the like. A limited number of “standard” components can then be made to meet the requirements of a generic population of patients. In this method, a non-invasive imaging scan, such as an X-ray or MRI, together with knowledge of the patient's genetic make-up, general body type, extent of the disease, degeneration, or trauma and the like, enables the surgeon to select components of the correct size and shape from the library for the patient.

In another method, each patient can receive one or more components that are custom tailored for the individual by producing a contour plot of the femoral and tibial mating surfaces and the size of the meniscal cavity. Such a contour plot may be constructed from imaging data (i.e., X-ray or MRI data) by a suitable computer program. From the contour plot, the correct surface geometry of the tibial and mensical components can be determined from the shape of the respective tibial plateau and femoral condyle and the orientation between the two surfaces in extension.

In accordance with the present invention, it has been discovered that the amount of varus deformity is a non-invasive method for determining the necessary thickness of the prosthetic system 100 required for proper functioning. Viewing a weight bearing anteroposterior X-ray, a cut and paste of the line drawn through the femoral condyles and repositioned to put them once again parallel to the tibial plateaus can yield a measurement for the approximate thickness of the prosthetic system 100. However, typically the proper thickness of the prosthetic system 100 may additionally be determined intra-operatively.

The prosthetic system 100 according to the present invention may be introduced by arthroscopically assisted implantation, typically via a 3 cm to 5 cm medial parapatella incision. The natural meniscus may be maintained in position or may be wholly or partially removed, depending upon its condition. Under ordinary circumstances, pieces of the natural meniscus which have been torn away can be removed, and damaged areas may be trimmed as necessary. In somewhat rarer instances, the entire portion of the meniscus residing in the meniscal cavity may be removed or is not present. If significant tibial bone loss (>0.5 mm) is found to be present, the surrounding cartilage and subchondral bone can be removed to provide a stable, relatively flat tibial surface. In some cases, it may be found necessary to remove all or a portion of the tibial spine as well. The tibial plateau can be resected to a more flat configuration by cutting, in a minimal fashion, along the sagittal plane, similar to the method used in a TKR or UKR, but typically only to a depth of less than 5 mm and possibly to a depth of only 1-3 mm, allowing the tibial component to bridge the defect. The tibial component can then be affixed to the tibial plateau, and the mensical component inserted proximal to the tibial component.

Prosthetic system 100 according to the present invention may also be used in conjunction with ACL or PCL repair, tibial osteotomy or articular surfacing procedures such as cartilage transplantations or abrasion anthroplasty. Following insertion of the prosthetic system 100, X-ray, fluoroscopy, or MRI may be used to assess the correct positioning of the prosthetic system 100 both intraoperatively as well as postoperatively. Since the surgical procedures used are not severe or irreversible, the modular prosthetic system 100 according to the present invention allows an unsuitable mensical component to be readily removed and replaced, either with a different mensical component from the library or by a custom mensical component.

FIG. 16 illustrates a prosthetic system 100 according to the present invention positioned in a medial compartment of a right knee joint 200 between a femur 202, including the femoral condyles 204, and a tibia 206 including the tibial plateau 208. The femur 202 and tibia 206 include interconnecting collateral ligaments 210. It is understood that prosthetic system 100 may just as easily be implanted in a lateral compartment or in the left knee of a patient or in any other body joint.

Following any necessary bone resection, one surgical procedure which may be used to implant prosthetic system 100 according to the present invention includes the following steps:
1. Verify preoperative indications:
   a. Valgus determination of <5° with erect anterior/posterior X-ray;
   b. Medial compartment disease only. Some lateral spurs may be present; and
   c. Pre-operative sizing via medial/lateral template measurement of anterior/posterior X-ray.
2. Standard Arthroscopy surgical prep:
   a. Infiltrate knee with Lidocaine/Marcaine and Epinephrine.
3. Arthroscopy:
   a. Inspect lateral patello-femoral compartments for integrity, some mild arthosis is acceptable;
   b. Removal of medial meniscus toward the rim along the anterior, medial and posterior portions;
   c. Initial arthroscopic osteophyte removal via ⅛″ osteotome and burr to allow for valgus positioning of the knee;
   d. Complete the removal (to the rim) of the posterior and posterior-lateral meniscus; and
   e. Confirm sizing of prosthetic system by measuring distance from resected meniscus to remaining anterior meniscus.
4. Medial parapatellar arthrotomy (mid-patella to tibial joint line).
5. Complete removal of visible osteophytes along the medial femoral condyle.
6. Insert thickness gauge and size for thickness of prosthetic system.
7. Insert and fix tibial component of prosthetic system.
8. Insert trial mensical component proximal to tibial component:
   a. Flex knee to approximately 50+degrees to fully expose the distal portion of the femoral condyle;
   b. Insert trial mensical component; and
   c. While applying insertion pressure, apply valgus stress to the tibia and “stretch-extend” the tibia over the trial mensical component.
9. Check for proper sizing with “true lateral” and anterior/posterior fluoroscope images of the knee while in extension:
   a. Ideally, the prosthetic system should be within 1 mm of the anterior/posterior boundaries of the tibial plateau and superimposed over the medial boundary.
10. Remove trial mensical component and flush joint with saline.
11. Insert the appropriate mensical component.
12. Confirm proper placement and sizing of prosthetic system with fluoroscopic images as with trial mensical component.
13. Maintain leg in extension and close wound after insertion of a Hemovac drain.
14. Place leg in immobilizer prior to patient transfer.

While embodiments of the invention have been illustrated and described, it is not intended that these embodiments illustrate and describe all possible forms of the invention. The words used in the specification are words of description rather than limitation, and it is understood that various changes may be made without departing from the spirit and scope of the invention.

1. A prosthetic system for implantation into a knee joint compartment between a femoral condyle and its corresponding tibial plateau, the prosthetic system comprising:
a tibial component arranged to be fixed to the tibial plateau and having a top face including a generally flat surface and an opposed bottom face; and
a mensical component having a generally elliptical shape in plan, the mensical component having a top face and an opposed bottom face including a generally flat surface, the meniscal component bottom face arranged to engage the tibial component top face to form the assembled prosthetic system, wherein the meniscal component is movable with respect to the tibial component.

2. The prosthetic system according to claim 1, wherein the mensical component is self-centering within the knee joint compartment.

3. The prosthetic system according to claim 1, wherein the mensical component top face includes a generally concave surface.

4. The prosthetic system according to claim 1, wherein the mensical component top face includes a generally convex surface.

5. The prosthetic system according to claim 1, wherein the tibial component further includes a guide extending upwardly from the top face thereof.

6. The prosthetic system according to claim 1, wherein the tibial component further includes an aperture provided therein arranged to receive a fastener for fixing the tibial component to the tibial plateau.

7. The prosthetic system according to claim 1, wherein the mensical component top face is generally non-conformal to the corresponding femoral surface.

8. The prosthetic system according to claim 1, wherein the mensical component top face is generally conformal to the corresponding femoral surface.

9. The prosthetic system according to claim 1, wherein the tibial component includes a peripheral edge joining the top and bottom faces thereof and including a first side, and the mensical component includes a peripheral edge joining the top and bottom faces thereof and including a first side, each peripheral edge being generally rounded.

10. The prosthetic system according to claim 9, wherein the tibial component first side is generally straight.

11. The prosthetic system according to claim 9, wherein the mensical component first side is generally straight.

12. The prosthetic system according to claim 9, wherein the peripheral edge of the meniscal component includes a first side, a second side opposite the first side, a first end and a second end opposite the first end, wherein a first dimension D is defined by the first end and the second end, and a second dimension F is defined by the first side and the second side, wherein the dimension F is from about 0.25D to about 1.5D.

13. The prosthetic system according to claim 1, wherein the tibial component is generally semicircular in plan.

14. The prosthetic system according to claim 1, wherein at least one of the tibial component and the mensical component is constructed of a biocompatible material selected from the group consisting of ceramics, metals, metal alloys, reinforced and non-reinforced thermoset or thermoplastic polymers, or composites thereof.

15. The prosthetic system according to claim 1, wherein at least one of the tibial component and the mensical component includes a portion made of a relatively low modulus material.

16. The prosthetic system according to claim 15, wherein the relatively low modulus material is selected from the group consisting of reinforced and non-reinforced elastomeric polymers, viscous-elastic materials, and hydrogels.

17. The prosthetic system according to claim 1, wherein at least one of the tibial component and the mensical component includes a biocompatible polymer capable of containing living cells.

18. The prosthetic system according to claim 1, further comprising a surface coating applied to at least one of the tibial component and the mensical component.

19. The prosthetic system according to claim 1, wherein at least one of the tibial component and the mensical component includes a biologically active material associated therewith.

20. A modular prosthetic system for implantation into a knee joint compartment between a femoral condyle and its corresponding tibial plateau, the prosthetic system comprising:
a standard tibial component arranged to be fixed to the tibial plateau and having a top face including a generally flat surface and an opposed bottom face; and
a plurality of different mensical components each having a generally elliptical shape in plan, the plurality of mensical components each having a top face and an opposed bottom face including a generally flat surface, wherein engagement of the bottom face of a selected one of the plurality of mensical components with the top face of the standard tibial component forms the assembled prosthetic system, wherein the meniscal component is movable with respect to the tibial component.

21. The prosthetic system according to claim 20, wherein the mensical component top face includes a generally concave surface.

22. The prosthetic system according to claim 20, wherein the mensical component top face includes a generally convex surface.

23. The prosthetic system according to claim 20, wherein the tibial component further includes a guide extending upwardly from the top face thereof.

24. The prosthetic system according to claim 20, wherein the tibial component includes a peripheral edge joining the top and bottom faces thereof and including a first side, and the mensical component includes a peripheral edge joining the top and bottom faces thereof and including a first side, the tibial component first side and the meniscal component first side each being generally straight.

25. A method for implantation of a prosthetic system into a knee joint compartment between a femoral condyle and its corresponding tibial plateau, the method comprising:
providing a tibial component having a top face including a generally flat surface and an opposed bottom face;
providing a mensical component having a generally elliptical shape in plan, the mensical component having a top face and an opposed bottom face having a generally flat surface;
surgically exposing the knee joint compartment;
inserting the tibial component into the knee joint compartment;
affixing the tibial component to the tibial plateau; and
inserting the mensical component into the knee joint compartment proximal to the tibial component such that the mensical component bottom face engages the tibial component top face to form the assembled prosthetic system, the meniscal component movable with respect to the tibial component.

26. The method according to claim 25, further comprising altering the condition of tissue located in the knee joint compartment.

27. The method according to claim 25, further comprising determining a required size and shape of at least one of the tibial component and the mensical component by examination of the knee joint.

28. The method according to claim 27, wherein the examination includes one or more of X-ray imaging and MRI imaging.

29. The method according to claim 25, further comprising selecting the mensical component from a library of mensical components of standard shapes and sizes.

30. The method according to claim 25, further comprising generating a custom mensical component whose size and shape are at least partially based on an examination of the knee joint.
